(19) United States
(12) Patent Application Publication
(10) Pub. No.: US 2006/0293517 A1
(43) Pub. Date: Dec. 28, 2006

(54) Enantiomerically pure cilazapril, process for preparation

(76) Inventors: Yatendra Kumar, Haryana (IN); Mohan Prasad, Haryana (IN); Kaptan Singh, Uttar Pradesh (IN); Kintali Venkata Ramana, Haryana (IN); Surendra Kumar Dhingra, New Delhi (IN)

Correspondence Address: RANBAXY INC., 600 COLLEGE ROAD EAST, SUITE 2100, PRINCETON, NJ, 08540, US

(21) Appl. No.: 10/547,821

(22) PCT Filed: Mar. 8, 2004
(86) PCT No.: PCT/IB04/00633

§ 371 Date: Aug. 7, 2006

(30) Foreign Application Priority Data
Mar. 6, 2003 (IN) 236/DEL/2003

(51) Int. Cl. A61K 31/5513; C07D 487/04

(52) U.S. Cl. 540/500; 514/221

(57) ABSTRACT

The invention relates to enantiomerically pure cilazapril and a process for preparing enantiomerically pure cilazapril. The invention also relates to pharmaceutical compositions that include the enantiomerically pure cilazapril and use of said compositions for treating a patient in need of an antihypertensive agent.

### FIELD OF THE INVENTION

The field of the invention relates to enantiomerically pure cilazapril and a process for preparing enantiomerically pure cilazapril. The invention also relates to pharmaceutical compositions that include the enantiomerically pure cilazapril and use of said compositions for treating a patient in need of an antihypertensive agent.

### BACKGROUND OF THE INVENTION

Chemically, cilazapril is 9(S)-[1-(S)-(Ethoxycarbonyl-3-phenylpropylamino]octahydro- 10-oxo-6H-pyridazo[1,2-a][1,2]-diazepine-1(S)-carboxylic acid and has the following structural Formula. It is disclosed in U.S. Pat. No. 4,512,924.

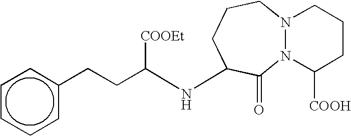

Because of the asymmetric carbon atoms in the above structure, it can exist in different stereoisomeric forms. These compounds can be, for example, racemates or optically active forms and may exist in either of the (S) or (R) enantiomeric forms. However the marketed form of the above structure (cilazapril) has (S) configuration at each asymmetric carbon atom. The enantio-purity or optical purity of cilazapril may be conventionally defined in terms of percent enantiomeric excess (% ee) which is the percent of the major enantiomer minus the percent of the minor enantiomer. A racemic mixture has an enantiomeric excess of zero.

Cilazapril is an angiotensin converting enzyme (“ACE”) inhibitor, and inhibits the formation of angiotensin II from angiotensin I via inhibiting the angiotensin converting enzyme, thereby reducing the systolic and diastolic blood pressure.

The strategy adopted in the prior art for the preparation of cilazapril comprises of treating appropriately bicyclic amine intermediate with protected carboxylic group with the suitable addendum, followed by hydrolysis to give the final product (cilazapril).

The prior art approach is not suitable from commercial point of view because the final product is always accompanied by the other enantiomeric impurities. In order to get the desired isomer, the final product requires purification by tedious and cumbersome purification processes such as column chromatography, HPLC or other techniques, thus making the approach commercially difficult to implement.

To achieve a high efficiency of reaction for industrial scale synthesis of cilazapril, it is necessary to minimize the enantiomeric impurities, and get the desired isomer with high yields and high optical purity.

Thus, the present invention provides a process which results in enantiomerically pure cilazapril. The choice of solvents has been found to be important for obtaining the pure product. The process of the present invention avoids purification by tedious and cumbersome processes such as column chromatography. The process of the present invention reduces the impurity content of the final product, eliminates the costly and time-consuming purification steps. The process economics are further improved owing to the higher yields of desired isomer.

### SUMMARY OF THE INVENTION

In one general aspect there is provided an enantiomerically pure cilazapril.

In another general aspect there is provided enantiomerically pure cilazapril which has a purity of more than 98% as determined by HPLC.

In another general aspect there is provided a pharmaceutical composition that includes a therapeutically effective amount of enantiomerically pure cilazapril; and one or more pharmaceutically acceptable carriers, excipients or diluents.

In another aspect there is provided a process for the preparation of enatiomerically pure cilazapril. The process includes obtaining a solution of crude cilazapril in one or more solvents; and recovering the enantiomerically pure cilazapril by the removal of the solvent.

The solvent may be one or more of lower alkanol, water, or mixtures thereof. The lower alkanol may include one or more of primary, secondary and tertiary alcohol having from one to six carbon atoms. The lower alkanol may include one or more of methanol, ethanol, denatured spirit, n-propanol, isopropanol, n-butanol, isobutanol and t-butanol. In particular, the lower alkanol may include one or more of methanol, ethanol, and denatured spirit. Removing the enantiomeric pure cilazapril may include one or more of distillation, distillation under vacuum, filtration, filtration under vacuum, decantation and centrifugation.

The process may include further drying of the product obtained.

The process may produce the enantiomerically pure cilazapril having a purity of more than 98% as determined by HPLC. The enantiomerically purity is more than 99.0%, for example, more than 99.5% or more than 99.8% as determined by HPLC using Chiracel ® OD-R column (10μm, 250 mm×4.6 mm).

In one general aspect, the solution of crude cilazapril may be obtained by heating the solvent containing crude cilazapril. It may be heated from about 30° C. to about reflux temperature of the solvent used, for example from about 30° C. to about 100° C. In particular, it may be heated from about 40° C. to about 60° C. It may be heated from about 15 minutes to about 10 hours. More particularly, it may be heated for about 2-3 hours.

In one general aspect the solution containing the crude cilazapril may be treated with charcoal before removing the solvent. The charcoal treatment may be carried out under heating conditions or it may be carried out at a lower temperature.

In another general aspect the slurry containing the product may be cooled prior to isolation to obtain better yields of the enantiomercally pure cilazapril and the product may be washed with a suitable solvent.

In another general aspect there is provided a method of treating hypertension using therapeutically effective amount of the enantiomerically pure cilazapril.

The details of one or more embodiments of the inventions are set forth in the description below. Other features, objects and advantages of the inventions will be apparent from the description and claims.

### DETAILED DESCRIPTION OF THE INVENTION

The inventors have developed an efficient process for the preparation of enantiomerically pure cilazapril, by obtaining a solution of crude cilazapril in one or more solvents; and isolating the enantiomerically pure cilazapril. The inventors also have developed pharmaceutical compositions that contain the enantiomerically pure form of the cilazapril, in admixture with one or more solid or liquid pharmaceutical diluents, carriers, and/or excipients. These pharmaceutical compositions may be used for treating a patient in need of antihypertensive therapy.

The crude cilazapril may be prepared by the methods known in the literature. In particular, it may be prepared using the reactions and techniques described in J. Chem. Soc. Perkin Transaction II, 1986; 747-755; J. Chem. Soc. Perkin Transaction I, 1979; 1451-1454, which are incorporated herein as reference.

In general, the solution of crude cilazapril may be obtained by dissolving crude cilazapril in a suitable solvent. Alternatively, such a solution may be obtained directly from a reaction in which cilazapril is formed. The solvent containing crude cilazapril may be heated to obtain a solution. It can be heated from about 30° C. to about reflux temperature of the solvent used, for example from about 30° C. to about 100° C. In particular, it can be heated from about 40° C. to about 60° C. It can be heated from about 15 minutes to about 10 hours. More particularly, it can be heated for about 2-3 hours. The product may be isolated from the solution by a technique which includes, for example, filtration, filtration under vacuum, decantation, and centrifugation.

The term “suitable solvent” includes any solvent or solvent mixture in which crude cilazapril is soluble, including, for example, lower alkanol, water and mixtures thereof. Examples of alkanol include those primary, secondary and tertiary alcohols having from one to six carbon atoms. Suitable lower alkanol solvents include methanol, ethanol, denatured spirit, n-propanol, isopropanol, n-butanol, isobutanol and t-butanol.

In one aspect, the solution containing crude cilazapril can be treated with activated carbon and filtered while hot or the slurry containing the enantiomerically pure cilazapril may be cooled prior to filtration.

In another aspect, additional or another solvent can be added to the clear solution to precipitate the enantiomerically pure cilazapril.

The product obtained may be further or additionally dried to achieve the desired moisture values. For example, the product may be further or additionally dried in a tray drier, dried under vacuum and/or in a Fluid Bed Drier.

Methods known in the art may be used with the process of this invention to enhance any aspect of this invention. For example, the solution containing the crude cilazapril may be heated for dissolution, or may be cooled to separate out the product or the slurry may further be cooled prior to filtration or the solution may be seeded with seed crystals of the product to enhance precipitation of the product.

The present invention has been described in terms of its specific embodiments certain modifications and equivalents will be apparent to those skilled in the art and are intended to be limited within the scope of the present invention.

### EXAMPLE 1

Preparation of Cilazapril

To a mixture of tertiary butyl 9(S)-(ethoxycarbonyl)-3-phenylpropylamino] octahydro-10-oxo-6H-pyridazo [1,2-a][1,2] diazepine-1(S)-carboxylate (10 g) and ethanol (100 ml), hydrazine hydrate (3 g) was added. The reaction mixture was stirred at room temperature for about 1 to 1.5 hours, till the completion of the reaction. The reaction mixture was concentrated under vacuum at 35-40° C. to get an oily residue. Acetic acid (2M, 100 ml) was added to the residue and stirred at room temperature for 4 to 5 hours. The residue was filtered and washed with water (50 ml). The pH of the mother liquor was adjusted to about 8.0 with 2N sodium hydroxide solution (100 ml) at 25-30° C., and was extracted with methylene chloride (50 ml ×2). The Organic layer was dried over anhydrous sodium sulphate (2 g) and concentrated under vacuum at 40-45° C. to get an oily residue (5.4 g).

To a mixture of the above oily residue (15 g) and toluene (75 ml), ethyl (+)-R-2-(4-nitorbenzene sulfonyloxy-4-phenyl butyrate (27.4 g) and N-methylmorpholine (6.4 g) was added and heated at 75 to 80° C. for 15 hours. The mixture was cooled to 25° C. and water (45 ml) was added to it. The pH of the reaction mixture was adjusted to about 8.8 with 2N sodium carbonate solution at 25-30° C. The organic layer was washed with water (45 ml ×2) and was dried on anhydrous sodium sulphate (5 g). The solution was concentrated under vacuum at 40 to 45° C. to get an oily residue.

The oily residue was dissolved in methylene chloride (75 ml) at room temperature and cooled to −5° C. Dry hydrochloride gas was passed into it at −5° to 0° C. for 6 hours. It was concentrated under vacuum at 30° to 35° C. to get an oily residue. The oily residue was dissolved in a mixture of water (105 ml) and diethyl ether (105 ml). The aqueous layer was separated out and pH was adjusted to 4.4 with 10% aqueous sodium hydroxide solution. The resulting mixture was extracted with methylene chloride (100 ml ×2). The organic layer was concentrated under vacuum at 40° C. to get an oily residue, which was dissolved in ethanol (50 ml) and heated to 40°-45° C. The oily residue so obtained was treated with activated carbon (1.0 g), filtered through a hyflo bed and the hyflo bed was washed with ethanol (10 ml). The solvent was concentrated under vacuum to about 20 ml of volume. Water (50 ml) was charged slowly at 45° to 50° C. in 1 hour to get a white precipitate. The reaction mixture was cooled to 35° C. and stirred for 2 hours and filtered. The white precipitate was washed with a mixture of cold ethanol and water (20: 80 v/v, 20ml). The wet material was dried at 30° C. to get crude cilazapril (10 g); Yield: 86.73%

### EXAMPLE 2

Purification of Cilazapril

To the crude cilazapril (25 g) obtained from Example 1, ethanol (125 ml) was added and heated to 40°-45° C. to get a clear solution. Activated carbon (2.5 g) was charged and stirred for 30 minutes at the same temperature. The reaction mixture was filtered through a hyflo bed and washed with ethanol (50 ml). The mother liquor was concentrated under vacuum at 40°-45° C. to about 2.5 to 3 times. Water (75 ml) was added slowly at 45°-50° C., to get a white precipitate in about 1 hour. The reaction mixture was stirred for 2 hour at same temperature, and cooled to 35° C., followed by stirring at 30° to 35° C. for another 2 hours. The white precipitate was filtered and washed with a mixture of water and ethanol (80: 20 v/v, 50 ml). The precipitate was kept under suction at room temperature for 10 minutes and dried under vacuum at 25 to 30° C. till water content was less than 5% to give enantiomerically pure cilazapril.

Yield : 85%

Quality: >98 by HPLC

Enantiomeric Purity: 99.99% by HPLC

While the present invention has been described in terms of its specific embodiments, certain modifications and equivalents will be apparent to those skilled in the art and are intended to be included within the scope of the present invention.

We claim:

1. A process for the preparation of enantiomerically pure cilazapril, the process comprising obtaining a solution of crude cilazapril in one or more solvent; and recovering the enantiomerically pure cilazapril by the removal of the solvent.

2. The process of claim 1, wherein the solution of crude cilazapril is obtained by heating the solvent.

3. The process of claim 2, wherein the heating temperature ranges from about 30° C. to about 100° C.

4. The process of claim 3, wherein the heating temperature ranges from about 40° C. to about 60° C.

5. The process of claim 1, wherein the solvent comprises one or more of lower alkanol, water, or mixtures thereof.

6. The process of claim 5, wherein the lower alkanol comprises one or more of primary, secondary and tertiary alcohols having from one to six carbon atoms.

7. The process of claim 2, wherein the lower alkanol comprises one or more of methanol, ethanol, denatured spirit, n-propanol, isopropanol, n-butanol, isobutanol, and t-butanol.

8. The process of claim 2, wherein the lower alkanol comprises one or more of methanol, ethanol, and denatured spirit.

9. The process of claim 1, wherein removing the solvent comprises one or more of distillation, distillation under vacuum, filtration, filtration under vacuum, decantation, and centrifugation.

10. The process of claim 1, further comprising additional drying of the product obtained.

11. The process of claim 1, further comprising forming the product obtained into a finished dosage form.

12. A method of treating hypertension, the method comprising providing a dosage form that includes enantiomerically pure cilazapril prepared by the process of claim 1.

13. Enantiomerically pure cilazapril having a purity of more than 98.0% by HPLC.

14. Enantiomerically pure cilazapril having a purity of more than 99.0% by HPLC.

15. A pharmaceutical composition comprising a therapeutically effective amount of enantiomerically pure cilazapril; and one or more pharmaceutically acceptable carriers, excipients or diluents.

16. A method of treating hypertension, the method comprising providing a dosage form that includes enantiomerically pure cilazapril.
